(19) United States
(12) Patent Application Publication
(10) Pub. No.: US 2016/0160262 A1
(43) Pub. Date: Jun. 9, 2016

(54) TEST KITS INCLUDING ACTIVATED MEDIATORS FOR DETECTING ANALYTES

(71) Applicant: Truthound LLC, Seal Beach, CA (US)

(72) Inventors: Aaron Michael Harry, San Diego, CA (US); Robert Crockett, San Luis Obispo, CA (US)

(21) Appl. No.: 14/562,672

(22) Filed: Dec. 6, 2014

(51) Int. Cl. C12Q 1/26; G01N 21/78

(52) U.S. Cl. CPC C12Q 1/26; G01N 21/78; G01N 2021/7759

(57) ABSTRACT

Activated mediators for detecting predetermined analytes in a sample, as well as kits including testing surfaces incorporating an enzyme-activated mediator are disclosed. The enzyme-activated mediator may have a first color prior to the application of a sample to the portion of the testing surface including the enzyme-activated mediator. The enzyme-activated mediator is configured to undergo a detectable color change to a second color different from the first color in response to the sample applied to the testing surface including a predetermined analyte reactive with the enzyme-activated mediator. Methods for making the testing surfaces and using the testing surfaces to detect analytes are also disclosed.

### FIELD

Methods, devices, and systems for detecting analytes in samples are described, and in particular, methods, devices and systems for detecting analytes in samples using activated mediators.

### BACKGROUND

Systems for detecting analytes including mediators specific to the analytes of interest are known. In such mediator systems, the detection reaction typically includes both the reaction where the mediator is activated by an appropriate enzyme and the reaction where the activated mediator is reacted with the analyte of interest. One problem with such systems is that the radicals generated by the enzyme that activates the mediator often destroy protein residues within the enzyme itself. This may cause a decrease in enzymatic activity as the number of radicals increase. For example, it has been shown that when there is an abundance of substrate present in a solution, there is sufficient dissipation of the radicals and enzyme activity is preserved. Unfortunately, as the reaction proceeds and the substrate is converted, the number of radicals becomes too great the reaction cannot come to completion even if more enzyme is added. Since the enzymes are expensive and hard to manufacture, it then becomes costly to use such a system for industrial and commercial processes.

Additional challenges that arise when developing assays for commercial or industrial processes (or any other process) involve preservation of the enzyme for long enough to be useful in catalyzing the activation of the mediator when the assay is run. The preservation of the enzymes often involves immobilization or other expensive and difficult processes. Another challenge is that typically, physical separation of the enzyme and the inactive mediator is required to prevent the reaction between the enzyme and the mediator from occurring prior to the start of the assay. Thus, in cases of colorimetric detectors or assays involving enzymes, preservation, immobilization and physical separation may add complexity and cost to the design of the assay.

Another disadvantage of the conventional assays is that many assays require a controlled sequencing of reactions through the use of multiple membranes and/or precisely engineered diffusion systems and/or multiple manual or automated assay steps. All of these methods of controlling the sequencing of reactions add cost and complexity to the assay design and use.

Another disadvantage of the conventional assays is that they often employ expensive analytical machines and software applications that practically make such assays prohibitive for home use. For example, multiplexing is commonly used in the biotechnology industry as a method for detecting one or more analytes of interest while using complex kinetic comparisons and calculations to determine the exact amount of molecule or molecules present when compared to standard curves. The chemical environment of the reaction mixture is often unknown and thus there must be constant comparison to controls. Most consumers do not have access to the spectrophotometers or the graphing and curve fitting software to utilize multiplexing in the home or field. In addition, the conventional assays do not generate their results within a matter of seconds, but typically generate their results in 5, 40, 60, or even 90 minutes, making such assays applicable to a laboratory environment, but again making such assays incompatible with home use, where it would be desirable to detect the presence or absence of an analyte of interest in the sample instantaneously.

Accordingly, what is needed is an assay that is low cost, has a simple design, is easy to use, and quickly provides a result output that is easily visible and understandable by the users.

### SUMMARY

The present invention satisfies such a need. The methods, devices, and systems described herein advantageously simplify the analyte detection assay process while providing a degree of accuracy similar to, or better than, competitive binding assays such as enzyme-linked immunosorbent assay (ELISA), without the need for multiple steps, extensive sample and reagent preparation, expensive machines, or complex analysis. Further, the testing methods described herein provide for assay testing that may be packaged for home use and that is able to detect the presence of a variety of different types of analytes, since the methods and systems described herein are versatile and easily configurable with appropriate reagents and testing conditions specific to detection of a wide variety of analytes of interest.

In one embodiment, a method for detecting one or more predetermined analytes in a sample includes providing at least one activated mediator configured to produce a detectable change upon reaction with the one or more predetermined analytes in the sample.

The method may further include combining one or more enzymes with one or more mediators to form an aqueous solution; and reacting the one or more enzymes with the one or more mediators to form an aqueous solution including the at least one activated mediator in radical form.

The method may include inactivating the one or more enzymes contained in the aqueous solution including the one or more mediators in radical form prior to further reaction.

The method may include separating the one or more enzymes contained in the aqueous solution including the one or more mediators in radical form from the aqueous solution. In one form, the method may include drying the aqueous solution including the one or more mediators in radical faun onto a powder carrier. In another form, the method may include drying the aqueous solution including the one or more mediators in radical form into powder form.

In one approach, the method further includes incorporating the solution into a testing surface by depositing at least a portion of the aqueous solution including the at least one activated mediator onto a testing surface and drying the testing surface. The testing surface may, in one form, include a test strip produced from low lignin paper.

By one approach, the method further includes covering at least a portion of the testing surface with at least one protective material in the form of a film or foil to restrict and/or prevent exposure of the activated mediator to air, UV light, or the like.

In one form, the method includes applying a sample liquid to the portion of the testing surface including the activated mediator by swabbing the portion of the testing surface including the at least one activated mediator with a material at least in part wetted with the sample liquid.

The detectable change upon reaction of the at least one activated mediator with the one or more predetermined analytes may be in the form of one of a visual colorimetric indication and a detectable change in electrical properties. In one approach, the visible colorimetric indication in response to a reaction of the at least one activated mediator with the one or more predetermined analytes in the sample further includes generating a visible color change of the at least one activated mediator from a first color to a second color in response to detecting a predetermined minimum concentration of the one or more predetermined analytes in the sample. In another approach, the visible colorimetric indication in response to a reaction of the at least one activated mediator with the one or more predetermined analytes in the sample further includes generating a color of a first saturation, brightness, and hue when a concentration of the one or more predetermined analytes below a first threshold is detected, generating a color of a second saturation, brightness, and hue when a concentration of the one or more predetermined analytes above the first threshold and below a second threshold is detected, and generating a color of a third saturation, brightness, and hue when a concentration of the one or more predetermined analytes above the second threshold and below a third threshold is detected. In yet another approach, the visible colorimetric indication in response to a reaction of the at least one activated mediator with the one or more predetermined analytes in the sample further comprises generating a certain gradation in the saturation and/or brightness and/or hue of a color that corresponds to a relative concentration of the one or more predetermined analytes in the sample. In yet another approach, the visible colorimetric indication in response to a reaction of the at least one activated mediator with the one or more predetermined analytes in the sample further includes generating a color indicating to a user that one or more reagents on the testing surface failed to function properly.

In one exemplary form, the mediator is 2,2′-azino-bis(3-ethylbenzothiazoline-6-sulphonic acid) (ABTS), the enzyme is a laccase obtained from at least one of Trametes Versicolor, Pleurotus Ostreatus, Pleurotus Species, and a recombinant laccase, and the activated mediator is ABTS radical cation.

In another exemplary form, the activated mediator is ABTS radical cation and the one or more predetermined analytes is selected from a group consisting of chemical compounds, amino acids (proteins, peptides, antibodies), pathogens, hormones, nucleic acids, metals, and combinations thereof. In one approach, the one or more predetermined analytes is bisphenol A (BPA), and the ABTS radical cation changes color from green to red in response to reacting with the BPA in the sample.

In one approach, a testing surface for detecting a predetermined analyte in a sample is provided. The testing surface includes an enzyme-activated mediator applied onto at least a portion of the testing surface. The enzyme-activated mediator is visible on the at least a portion of the testing surface to a user in a first color prior to application to the at least a portion of the testing surface including the enzyme-activated mediator of a sample. The enzyme-activated mediator is configured to undergo visible color change to a second color different from the first color in response to the sample applied to the at least a portion of the testing surface including the enzyme-activated mediator including a predetermined analyte reactive with the enzyme-activated mediator.

In yet another approach, a kit for detecting a predetermined analyte in a sample is provided. The kit includes a testing surface including an enzyme-activated mediator applied onto at least a portion of the testing surface. The enzyme-activated mediator is visible on the at least a portion of the testing surface to a user in a first color prior to application to the at least a portion of the testing surface including the enzyme-activated mediator of a sample, the enzyme-activated mediator being configured to undergo a visible color change to a second color different from the first color in response to the sample applied to the at least a portion of the testing surface including the enzyme-activated mediator including a predetermined analyte reactive with the enzyme-activated mediator. The kit further includes a swab configured to absorb a portion of the sample and apply the absorbed portion of the sample to the at least a portion of the testing surface including the enzyme-activated mediator. The kit further includes a packaging material separating and enclosing the testing surface and the swab.

The methods, devices and systems described herein provide numerous advantages over the presently used systems and methods. One advantage is that the methods, devices and systems described herein provide a visual, color-based indication of the presence of the analyte of interest. Another advantage of the present methods, devices and systems is that easy to understand results of the assay are provided within seconds to a user as compared to at least 5 minutes for the fastest conventional tests. Another advantage of the methods, devices and systems provided herein is that assays may be incorporated onto a single testing surface and performed in a single detection step, greatly simplifying assay design and use compared to conventional assays. Another advantage is that the methods, devices and systems provided herein are easily configurable with reagents and activated mediators that may test for a presence of a variety of analytes. Yet another advantage of the methods, devices and systems described herein is that they have a significantly lower per-test cost than conventional units and do not require the users to acquire expensive equipment, making the methods, devices and systems described herein easily amenable to home use. Further advantages will be appreciated by those of ordinary skill in the art with reference to the following detailed description and claims.

### DETAILED DESCRIPTION

The methods and systems according to some embodiments described herein advantageously provide for the use of mediated enzyme assays to obtain a binary result visually, electronically, or through other methods of detection. In addition, the methods and systems according to some embodiments described herein advantageously permit the enzymatic processes of a mediated enzyme assay to be separated from the final assay, creating a final assay comprised of activated mediators. Separating the enzymatic processes from the final assay advantageously allows the assay to generate a result that is significantly faster than conventional assays. Additionally, separating the enzymatic processes from the final assay advantageously allows for a generalizable technique suitable for creating assays to detect a wide variety of analytes. Additionally, separating the enzymatic processes from the final assay advantageously allows the resulting assay to be dehydrated onto a single testing surface or carrier, providing a form which is both simple and highly stable for packaging into a consumer product.

In one embodiment, a kit for detecting one or more analytes is provided. As used herein, an “analyte” will be understood to mean any substance, the presence of which is being detected, or its levels being measured. Some exemplary analytes may be chemical compounds, amino acids (proteins, peptides, antibodies), pathogens, hormones, nucleic acids, metals, or the like.

The kit may include one or more activated mediators configured to detect the presence of one or more analytes in a sample. As used herein, “mediators” will be understood to mean any substance that modifies an enzyme's substrate specificity. As used herein, “activated mediators” will be understood to mean mediators which have been reacted with a compatible enzyme or enzymes to become activated in one or more of their radical states. The kit may include one or more mediators selected from a practically unlimited number of mediators specific to analytes the kit is designed to detect. The mediators may be previously activated and stabilized or preserved in solution or in dehydrated form. Without wishing to be limited by theory, the separation of the mediator from the enzyme(s) used to activate the mediator, and the preservation of the activated mediator in solution or in dehydrated form permits the activation of the mediator to take place separately from, and prior to the reaction of the activated mediator with the analytes during the assay, and thereby advantageously reduces the final assay time.

Generally, when enzyme-based assays are dependent on a mediator, the slowest part of the reaction is the enzymatic reaction. The inventors have found that if the enzymatic reaction and the reaction between the activated mediator and the analyte are separated, then the relatively fast reaction between the activated mediator and the analyte may be used to engineer near instantaneous (accurate result within 30 seconds, e.g., about 1-5 seconds in one approach, about 5-10 seconds in another approach, about 10-20 seconds in another approach, and about 20-30 seconds in yet another approach,) detection of various analytes compared to the 5 minute or more duration of the test in other conventional enzyme mediator systems. Further, the inventors have found that the methods described herein provide for development and customization of assays suitable for consumer use without an accompanying loss of detection limits, specificity, and accuracy as compared to the conventional full enzyme mediator system based tests. Further, the inventors have found that the activated mediator may be preserved in an environment that is stable for extended period of time, enabling the final assay to be simplified down to one detection step that identifies the presence of the analytes in question.

While the present application describes an exemplary reaction of a single mediator with a single enzyme, those skilled in the art will recognize that the methods described herein may be extended to single mediators reacting concurrently or sequentially with multiple enzymes, or to multiple mediators reacting concurrently or sequentially with single or multiple enzymes.

In one approach, an activated mediator may be produced by reacting the native mediator with an enzyme in a suitable medium, for example, an aqueous solution. For example, the reaction between the mediator and the enzyme may be a redox reaction. Optionally, after the mediator is activated, the enzyme may be inactivated. In one approach, the enzyme may be inactivated by heating at a temperature effective to inactivate the enzyme. For example, the mixture of the activated mediator and enzyme may be heated to a temperature of between 50° C. and 85° C. for about 1 hour in order to inactivate the enzyme. It will be appreciated that the enzyme may also be inactivated by other suitable means, for example, chemically, enzymatically, via UV light, or the like. Alternately the enzyme may be left active in solution, or dehydrated along with the activated mediator, if it does not adversely interfere with later reactions.

After the optional inactivation of the enzyme the activated mediator may optionally be separated from the enzyme in solution, for example, by filtering. In one approach, the activated mediator may be strained from the cooled solution using a 0.2-0.45 μm micron filter of any type that does not contain any quantity of lignin, other polyphenols, phenols, hydroxylated benzene derivatives, diamines, or certain polycyclic aromatics, or antioxidant chemicals. It will be appreciated that the activated mediator may also be separated from the cooled solution by another suitable technique, for example, molecular sieves, chromatography (e.g. gel electrophoresis, column separation, etc.), or the like. It will also be appreciated that instead of separating the activated mediator from the solution, the enzyme may be separated from the aqueous mediator solution, for example, by filtering. In one approach, the separation of the activated mediator into a separate vessel and subsequent calculation of the number of the radicals of the activated mediator needed to fully oxidize an intended substrate (i.e., analyte of interest) provides for the reaction of the activated mediator and the analyte of interest to occur separately at a later time. It will be appreciated that optionally, the active or inactivated enzyme may be left in the solution containing the activated mediator, or dehydrated along with the activated mediator, if the active or inactivated enzyme does not adversely interfere with later reactions.

Without wishing to be limited by theory, since the radicals of the activated mediator may be remotely generated, they can be removed from the vessel including the enzyme before the concentration of the activated mediator radicals destroys the enzyme. Thus, large quantities of the activated mediator may be continuously produced for use in the mediated reactions with the analytes of interest without using large amounts of the enzymes, which advantageously results in a significant savings cost, since enzymes are expensive. In one approach, if the enzymes are attached to a solid material, the enzyme or enzymes may be removed from the solution by a physical process such as centrifugation.

The activated mediator optionally separated from the aqueous solution including the optionally inactivated enzyme may then be placed in a filtered aqueous solution. The filtered aqueous solution may be diluted with an alcohol-based solvent such as isopropyl alcohol or ethyl alcohol, or with a non-alcohol based solvent, to decrease the drying time, and/or to produce the desired color output when the activated mediator is reacted during the assay. Without wishing to be limited by theory, the presence of an alcohol of some type may be advantageous in some cases to shift the color produced by the reaction during the assay.

A portion of the activated mediator in the aqueous solution may then be incorporated in liquid form into a kit for detecting an analyte. In one embodiment, a kit may be provided to consumers in a self-containment or test device that may be in the form of a plastic/glass/acrylic tube or vial, or another enclosure such as a support structure including one or more covered wells that may contain the active components and protect the active components from ambient air and/or possible contamination. Optionally, the containment device may include a locking mechanism to prevent the device from unintentionally opening during the test such that the reagents and chemicals within the test device may not be accessed or contaminated after the sample of interest has been inserted into the device.

In another embodiment, a portion of the activated mediator in the aqueous solution may be incorporated onto a suitable carrier in order to create a dehydrated storable deposit of mediator for use in assays, or any other methods that may advantageously utilize a dehydrated activated mediator. Suitable carriers may include surfaces such as paper or polymers. Suitable carriers may also include a powdered carrier that is either water soluble (e.g. sodium chloride, glucose, sucrose) or water insoluble (e.g. silicon dioxide).

In yet another embodiment, a portion of the activated mediator in the aqueous solution may be dried to powder form. The resulting powder may then be included in a self-containment or test device such as that described previously, or stored for later rehydration and use.

In yet another embodiment, a portion of the activated mediator in the aqueous solution may be placed on a support carrier that may provide a testing surface, such as a membrane, a test strip, or the like. The testing surface may be made of a single-layer, or two or more laminated layers. In one approach, the testing surface is made of a material that is substantially free of chemicals that may inhibit the activated mediator or participate in the assay reaction, and is absorptive to enable the activated mediator to be evenly spread thereon. For example, acid and lignin free paper (i.e., paper that contains no more than 5% lignin, preferably no more than 1% lignin) may be an appropriate testing surface material. The testing surface with the activated mediator placed thereon may be dried at a temperature of about 26° C. to about 85° C. to dehydrate the activated mediator and secure the activated mediator onto the testing surface. The testing surface preferably provides sufficient surface area to allow adherence of the dried activated mediator onto the testing surface.

Optionally, the testing surface may include an adhesive material to facilitate the attachment of the dried activated mediator to the testing surface. Preferably, such an adhesive does not contain any chemical that affects the activity of oxidase enzymes or peroxidase enzymes, or affects the stability of the activated mediator. An acrylic-based pressure sensitive adhesive (PSA) is one example of a suitable adhesive.

The dried testing surface with the activated mediator thereon may be then covered with one or more protective materials to restrict and/or prevent exposure of the activated mediator to air, UV light, etc. The protective materials may be in the form of a film or foil and may advantageously inhibit the active mediator from undesirably reacting or degrading prior to the use of the testing kit, and advantageously extend the possible storage time of the testing surface prior to use in the assay. The protective material may be a polymer such as polyethylene, polypropylene, polystyrene, polycarbonate, or the like, or a metal such as aluminum in the form of a foil. Optionally, a mesh-like separator may be placed between the activated mediator and the protective material. The mesh-like separator may be a thin (<0.5 mm) sheet that allows water or a solution of water and other chemicals to pass through it and onto the activated mediator. Examples of suitable mesh-like separators include perforated plastic sheets or meshes comprised of interwoven metal or plastic filaments. It will be appreciated that the protective material or materials may be directly placed over the activated mediator without the use of a separator material.

In a preferred embodiment, a kit for detecting an analyte consists of an activated mediator, and a means for introducing a target sample to the activated mediator. The activated mediator can be either dispersed or dissolved in a solution contained in a test device (such as a tube or vial) or dehydrated onto a testing surface. The target sample can be either on a solid surface or in solution. If the target sample is on a solid surface, the kit can employ a moistened swab, wipe, or any suitable material to lift the target analytes from the surface of the sample being tested. Alternatively, the activated mediator in solution may be deposited onto the surface containing the target sample. If the target sample is in solution, the swab or other material may be moistened with the sample solution and introduced onto the activated mediator, or the sample solution may be physically deposited onto the activated mediator, for example, via one or more drops applied, for example, via a pipette into the test device containing the activated mediator.

In one approach, the target sample is combined into a solution configured for preparing the target analytes for analysis prior to introducing the target sample to the activated mediator. The solution may be configured by inclusion of one or more additives that may produce a desired result in the assay reaction. For example, additives to the solution can decrease or increase specificity of a chemical to the test, thereby allowing for multiple tests to be developed using the same basic system. Chemical compounds, proteins, nucleic acids, or other substances may be used to alter the specificity of the system.

The solution may also include additives to control the output color of the test indicator. For example, a first liquid may include a substance that would result a red color output indicating a presence of a particular analyte in the sample, a second liquid may include a substance that would result a green color output indicating a presence of a particular analyte, and a third liquid may include a substance that would result a blue color output indicating a presence of a particular analyte. In one form, the solution may include one or more additives that may react with the activated mediator to provide the desired coloration of the mediator and/or the solution and/or the testing surface. It will be appreciated that instead of including the color generating substances in the solution that includes the sample to be tested, the testing surface itself may include one or more substances configured to generate the desired output color upon detection of the analyte.

In another approach, the liquid used to lift the target analytes from the surface of the sample being tested may be chosen depending on the solubility or other properties of the analyte being detected. As an example, the liquid used to lift the target analytes from the surface of the sample being tested may contain solvents such as dimethylsulfoxide, isopropyl alcohol, or ethyl alcohol, or a solution of chemicals in water to increase the extraction of analytes which have low water solubility (e.g. bisphenol A and its derivatives, which have less than 50 ppm solubility in water).

In one embodiment, the liquid used to lift the target analytes from the surface of the sample being tested contains molecules that can form inclusion complexes with hydrophobic molecules. Molecules that are chains of dextrin which form a ring structure with a hydrophobic center and hydrophilic exterior (cyclodextrins) are preferred. (2-hydroxypropyl)-B-Cyclodextrin (HP-B-CD) is a more preferred embodiment because of its safety, molecular weight, and solvation property of hydrophobic molecules such as bisphenol A (BPA). Cyclodextrins can be used to improve the pickup or lifting of hydrophobic molecules from the testing surfaces. Using cyclodextrins as an additive in the liquid used to lift the target analytes from the surface of a sample being tested is extremely useful for ELISA and biosensors in the extraction phase, where currently there lacks a solubilizer that is effective, inexpensive, easy to use, non-interfering and safe.

In a preferred embodiment, the tip of a swab is saturated with a solution of a cyclodextrin such as (2-hydroxypropyl)-B-Cyclodextrin (HP-B-CD), along with a typical solvent to lift BPA from the surface of the sample being tested. In one approach, a solubilizer is configured such that once it comes in contact with an increased water percentage (such as is present in the body), it converts BPA into the original, insoluble form of BPA and therefore does not make the lifted BPA more dangerous to the user.

It will be appreciated that the liquid may be chosen depending on any other parameters appropriate for the particular assay being performed. As such, the liquid used to lift the target analytes from the surface of the sample being tested may or may not contain chemicals and/or buffers that may participate in the chemical reaction between the activated mediator and the analyte. For example, the liquid used to swab the sample surface may contain buffers, reagents, activators, modifiers, or any other chemical or chemicals that may serve as a solvent for the target analyte, modify the pH, or chemically modify and/or prepare the target analyte. As another example, the liquid may include modulators that control the detection limits, i.e., minimum concentration thresholds for the detection of any analyte such as a chemical compound, an amino acid, a metal, or other analyte of interest.

In a more preferred embodiment, a kit for detecting an analyte consists of an activated mediator dehydrated onto a testing surface, and a separate moistened swab, wipe, or other suitable material configured for lifting the target analytes from the surface of the sample being tested and optionally preparing them for analysis. After the swab is rubbed against the surface of the sample to be tested, the swab may then be used to transfer the target analytes onto a portion of the activated mediator contained on the testing surface. If the analyte is present, or if a certain concentration of the analyte is present, the activated mediator is configured to change color very quickly, for example in 1-2 seconds in one approach, 1-5 seconds in another approach, and 1-10 seconds in yet another approach, 1-20 in yet another approach, and 1-30 second in still another approach.

The appearance of a visible color indicator upon the completion of the biochemical reaction being performed during the assaying advantageously provides a reliable “binary” output system, whereby the biochemical reaction occurring in the assay is linked to an easily understandable color-based visual output that indicates to a user whether the analyte of interest is present in the tested sample. In a simple binary system, an indicator color either appears or disappears to indicate the presence or absence of an analyte of interest. In a more preferred binary system, red, or any other color, occurs when a sample being tested is positive for an analyte, or group of analytes, and green, or any other color, occurs when the sample does not contain the analyte(s). The color selection may be reversed if the analyte is desirable, such that green indicates a positive test and red indicates a negative test for analytes of interest. Advantageously, the binary output system as described herein provides a quick and easily understandable color-based result to a user without the need of sophisticated and expensive analytical machines to analyze the outputs of biochemical reactions.

As used herein, “color” will be understood to mean a visual indication with a specific saturation, brightness, and hue. It will be appreciated that the perceived appearance of a color is equivalent to the change from a first color to a second color, whereby the first color has a very low saturation and/or brightness and/or hue relative to the second color. Similarly, the perceived disappearance of a color is equivalent to the change from a first color to a second color, whereby the second color has a very low saturation and/or brightness and/or hue relative to the first color.

In one approach, a binary-type answer may be provided by the detection methods as described herein by using one or more predetermined chemicals either on a testing surface or in the liquid used to lift the target analytes from the surface of the sample being tested such that a predetermined threshold is set at which the assay solution will visibly change color due to a reaction with a specific analyte.

In another approach, the visible color indicator may involve more than two colors, created by the reaction of more than one activated mediator reacting with an analyte. As an example, a green color that develops to represent “safe,” a yellow color that develops to represent “caution,” and a red color that develops to represent “danger.”

In yet another approach, the visible colorimetric indication in response to a reaction of an activated mediator with one or more predetermined analytes in the sample comprises generating a certain gradation in the saturation and/or brightness and/or hue of a color that corresponds to a relative concentration of the one or more predetermined analytes in the sample.

It will be appreciated that the testing surface including the activated mediators may include one or more components that, upon detection of an analyte of interest, may generate variable outputs other than a visible color change, for example, differential charges which can be detected by an external electrical circuit.

As an example, an activated mediator could be used to simplify, and thus significantly reduce the cost, of electronic-based detection systems such as glucose monitors. Glucose monitors currently use enzymes to affect changes in substrates across specifically designed membranes, employing circuitry to analyze the changes that occur. In an embodiment of the present invention, a suitable activated mediator on a single testing surface will release electrons in the presence of an appropriate molecule that can be detected by electrodes or other appropriate circuitry.

Optionally, the form of the assay could be made such that values could be obtained using a spectrophotometer and calibrated to a standard curve. A spectrophotometer may thus be used to describe the kinetics of enzymes and/or study the activity of inhibitors, cofactors, modulators, reaction products, or other materials of interest. For example, the biochemical reaction used to detect one or more analytes of interest may be linked to a processor-based device running software that may include databases of enzymes and/or substrates, and may be configured to predict the likely enzyme/substrate interactions and the kinetics of the enzymes or multiplexes.

In one embodiment, a mediator such as 2,2′-azino-bis(3-ethylbenzothiazoline-6-sulphonic acid) (ABTS) may be activated using a type of laccase enzyme and may be used to test for a wide range of target substances such as, but not limited to, bisphenol A (BPA), bisphenol S (BPS), or the like polyphenolic and diamine compounds, anti-oxidants, simple phenols, lignin/lignin breakdown products, metals, and dihydroxybenzene and its derivatives, as well as the presence of proteins in a sample. Generally, ABTS is used to observe the reaction kinetics of specific enzymes, for example, in enzyme-linked immunosorbant assays (ELISA) to detect for binding of molecules to each other. ABTS is also used as a substrate with hydrogen peroxide for a peroxidase enzyme or alone with enzymes such as laccase or bilirubin oxidase. Without wishing to be limited by theory, ABTS may allow the reaction kinetics of peroxidases to be followed, and ABTS may be used to indirectly follow the reaction kinetics of any hydrogen peroxide-producing enzyme, or to simply quantify the amount of hydrogen peroxide in a sample.

Herbicides, pesticides, rodenticides, and other chemical compounds that have the appropriate structure can be detected quickly using ABTS radicals as the activated mediator. ABTS acts as both the chromatic substance and the detection substance thereby simplifying the detection of these chemicals in the environment or any sample of interest.

In a preferred embodiment, ABTS is used as an activated mediator for detecting an analyte of interest such as BPA in a sample of interest, for example, water. Specifically, a testing surface including activated ABTS (i.e., ABTS cation radical), which may be prepared as described above, is moistened with a solution containing the sample to be tested. Upon contact of the sample solution with the activated ABTS, the activated ABTS remains green on the testing surface unless BPA is present in the sample solution. In one approach, if a detectable amount of BPA is present in the sample solution, the activated ABTS may turn from green to a predetermined color, for example, red, to visually indicate to a user that BPA is present. In another approach, if a concentration of BPA exceeding a predetermined minimum threshold is present in the sample solution, the activated ABTS may turn from green to a predetermined color, for example, red, to visually indicate to a user that BPA is present. In still another approach, the color change resulting from the test may be a certain gradation of a color, representing a concentration of the detected BPA. In other words, the intensity of the red color may depend on the concentration of the detected BPA, i.e., the lower the concentration, the lighter the red color generated on the testing surface, and the higher the concentration of BPA, the deeper the red color generated on the testing surface.

Turning to the specifics of the method for detecting the presence of BPA using activated ABTS cation radical, in one preferred embodiment, one liter of activated ABTS cation radical is prepared by dissolving approximately 0.5 gram of laccase in 50 ml of super pure water and stirring for 30 minutes. The laccase may be obtained from Trametes Versicolor and/or from Pleurotus Ostreatus or Pleurotus Species. Appropriate recombinant versions of laccase could also be used. Depending on a specific application and analyte or analytes being tested for, two different forms of laccase may be used to alter the properties of the test reaction. As used herein, “super pure water” is taken to mean 18 MΩ water. However, it will be appreciated that water down to 10 MΩ may be acceptable. In one approach, reverse osmosis-deionized water with 0-5 ppm (5 mg/L) contaminant concentration is suitable for use instead of super pure water.

After the laccase is dissolved in the water and stirred, approximately 3.33 grams of ABTS is dissolved in 950 ml of super pure water and stirred for 5 minutes to form a light green solution. The laccase solution may or may not be filtered using a 0.2 um to 0.45 um vacuum filter prior to reaction with the ABTS solution. The 50 ml laccase solution is then slowly added to a stirring solution of ABTS until a dark green solution forms. This dark green solution is then stored at room temperature of about 20° C. to about 28° C. for approximately 48 hours to fully react. Optionally, the solution may be heated to 37-38° C. for 4 hours to increase the effective activity of the enzyme. More or less than 0.5 gram of laccase per liter of final solution, and more or less than 3.33 grams of ABTS per liter of final solution, may be used to achieve a desired final color intensity.

Without wishing to be limited by theory, when using an activated mediator to interact with a target substance to produce a measurable or detectable response, the mediator can often be activated inadvertently by exposure to contaminants in the ambient air. The activated mediator (e.g., ABTS) may be preserved for prolonged periods for time as follows. In one approach, after ABTS cation radicals are created using an activating enzymatic reaction (or another suitable technique), the activating enzyme may be deactivated, denatured, and/or removed, and the activated ABTS cation radicals may be preserved for prolonged periods of time in a 50-100 mM concentrated aqueous vessel that is sealed from ambient air. In one form, the pH of the radical-rich solution being preserved is stable when the pH is above 7. The pH of the solution may be buffered to a slightly basic pH (e.g. between 7-8), and the solution may contain any suitable additive such as simple alcohols (e.g. isopropyl, ethyl, methyl alcohol), or surfactants (e.g. tween20, triton X-100, PEG5000) that do not interfere with the chemistry (e.g. enzyme(s), mediator(s), reagent(s), etc.).

In one approach, a solution of activated ABTS cation radicals may be utilized in liquid form to interact with chemical compounds of interest to achieve a desired result using the methods, devices, and systems described herein. By means of example only, a solution of activated ABTS cation radicals may be incorporated in liquid form into a kit for detecting lignin and the breakdown products of lignin in paper, advantageously providing archivists with an easy way to determine the status of the documents in a collection and to choose safer paper for new archives. As another example, a solution of activated ABTS cation radicals may be used to physically remove lignin from a paper pulp solution by catabolism.

In another approach, a liquid solution of activated ABTS cation radicals may be prepared for downstream processes such as printing, stamping, inking, inkjet, or other method by optionally adding between 5% and 95% non-volatile preservative and/or thickener such as sucrose, cellulose, non-protein based gels, or the like, and/or an amount of a surface tension lowering chemical such as alcohols (e.g. isopropyl, ethyl, methyl alcohol, non-volatile alcohols such as fatty alcohols), or surfactants that do not interfere with the chemistry (e.g. tween20, triton X-100, PEG5000).

In one embodiment, a stable activated ABTS solution may be incorporated onto a suitable carrier in order to create a dehydrated storable deposit of mediator for use in assays, or any other methods that may advantageously utilize a dehydrated activated mediator. Suitable carriers may include surfaces such as paper or polymers. Suitable carriers may also include a powdered carrier that is either water soluble (e.g. sodium chloride, glucose, sucrose) or water insoluble (e.g. silicon dioxide).

In yet another approach, a liquid solution of activated ABTS cation radicals may be dried directly to powder form, without the use of a carrier, for later rehydration or use in dry form. It will be appreciated that any activated mediator other than ABTS cation radicals may be similarly dried directly to powder form and stored in a dehydrated form for later use.

In a preferred embodiment, a liquid solution of activated ABTS cation radicals may be incorporated into a kit containing a testing surface and a swab to transfer a sample to the testing surface. A preferred testing surface may be in the form of a test strip prepared from acid-free, low-lignin (e.g., no more than 5% lignin) paper commonly available as archival paper. The test strip is then saturated with the solution, preferably in its entirety, and dried. The test strip may then be cut into stickers, covered with a protective cover, and affixed onto the surface of a sealed “wet nap” style package. The wet nap style package may contain a swab in a pre-measured solution of water and 5%-95% alcohol such as isopropyl, ethyl, or methyl alcohol. In one approach, in the absence of an alcohol, the resultant color of ABTS radicals in the presence of BPA and its derivatives may be purple, while in the presence of an alcohol such as, but not limited to, isopropyl, ethyl, or methyl alcohol, the resultant color of ABTS radicals in the presence of BPA and its derivate may be red or orange, which may be advantageous as an easy-to-interpret warning color.

While ABTS is described in a preferred embodiment as an activated mediator for detecting BPA in a sample, it will be appreciated that the activated ABTS may be used to detect not only BPA, but other analytes of interest on a solid surface, in a water sample, or in another liquid sample that is not water, for example, in a juice, ice tea, or sports drink. With appropriate modifications, activated ABTS may be used to test for a wide range of target substances such as, but not limited to diphenylmethane derivatives such as bisphenol A (BPA), bisphenol B, bisphenol Z, or the like, BPA derivatives not derived from diphenylmethane such as bisphenol S, bisphenol P, and bisphenol M, simple phenols (1-hydroxylbenzene and its derivatives), dihydroxybenzenes and their derivatives, and polyphenolic molecules with an exemplary formula 1 as shown below:

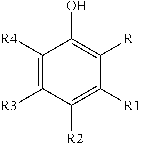

where R, R1, R2, R3, R4 can be all different or all the same and can be any number of substitutions including but not limited to hydrogen(s), halogen(s), hydroxyl(s), aryl group(s), alkyl group(s), carboxylic acid(s), other phenolic moieties, and many others. Some other target substances include diamine compounds, anti-oxidants, lignin/lignin breakdown products (themselves polyphenols that include the basic molecular structure represented by formula 1 above), as well as the presence of proteins in a sample. Select heterocyclic aromatic amines and select aromatic amines may also be a target substance.

Another set of target substances that could be detected with careful selection of mediators or modifiers may include those containing an azo group (azo dyes) with the basic structure R-N=N-R′ where R and R′ can be aryl or alkyl and the substitutions/derivatives of the appropriate groups. Yet another set of target substances may carry the name azine and have a core represented by the exemplary formula 2 below where R1, R2, R3 and R4 can be aryl or alkyl and the substitutions/derivatives of the appropriate groups (e.g., ABTS, Syringaldazine):

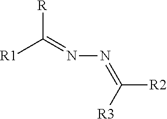

Additional target substances include any molecule or atom that disrupts the stability of radicals in aqueous solutions as well as molecules that bind to the active site of the enzyme, and molecules that destroy the structure of the protein or mediator. These interfering chemicals may include azide salts, proteases, and metal ions. Non-phenolic target molecules and mediators include, but are not limited to: N-hydroxy alcohol derivatives, violuric acid (VLA), 4-hydroxybenzyl alcohol (HBA), 1-hydroxybenzotriazole (HBT), N-hydroxyacetanilide (NHA), N-hydroxyphthaleimide (NHPI).

Yet another set of target substances that could be detected with careful selection of mediators or modifiers include, but are not limited to, peanut, gluten, grain, or other allergens in foods; hormones, antibodies and/or pathogens and/or DNA/RNA in blood and/or sputum and/or urine, and/or sweat; and for the presence of chemical compounds or contaminants in environmental samples, water, other drinkable beverages, foods, and the like.

In one example of an appropriate modification to a test utilizing activated ABTS, the activated ABTS solution may be used to accurately detect the presence of proteins in a sample, as indicated in some instances by a color change from green to purple upon reaction of the proteins with the activated ABTS, and in other instances by a reduction of intensity of green upon reaction of the proteins with the activated ABTS.

In another example of an appropriate modification to a test utilizing activated ABTS, the decolorization of activated ABTS upon interacting with substances that destabilize radicals, primarily heavy metals (increasing detection down and to the right of the periodic table) and some other molecules or ions that interfere with radical stability is utilized as the testing mechanism. An exemplary embodiment is an activated ABTS decolorization assay in combination with a stable red dye that is insoluble in a water-isopropyl alcohol mixture and is relatively impervious to degradation by ABTS radicals. In such an embodiment, the testing surface is first colorized with a red dye. The dye used is preferably Sudan Red 7B (N-Ethyl-1-((4-phenyldiazenyl)phenyl)diazenyl)naphthalen-2 -amine) but may be substituted with numerous other appropriate red dyes. Sudan Red 7B dissolved in acetone (or another appropriate solvent) is applied to the testing surface and the solvent is evaporated. The result is a red testing surface for the addition of aqueous activated ABTS with the green radical visually covering the red dye on the testing surface. This creates a visually green testing surface when dried. Upon interacting with heavy metals, the activated ABTS decolorizes, exposing the red dye underneath. Visually, this indicates to the user a test for heavy metals that is green in the absence of heavy metals and red in the presence of heavy metals. It will be appreciated that this modification of exposing static colors upon decolorization of an activated mediator, or the inverse of hiding a static color upon colorization of an activated mediator, is not limited to activated ABTS and can be extended to any activated mediator assay.

In yet another example of an appropriate modification to a test utilizing activated ABTS, a conjugated protein or nucleic acid conjugate could be used to create a testable complex. For example, a test for methyl benzene could utilize a protein that binds to methyl benzene and has many phenolic moieties, which would in turn react with activated ABTS.

In addition, an activated mediator other than ABTS may be used for detecting BPA, BPS, or other polyphenolic compounds in the sample of interest. It will be appreciated that the red output color is chosen by way of example only, and that, depending on the substances chosen by a manufacturer of the testing kit and the desired output colors, the activated ABTS may turn to any desired color (e.g., yellow, blue, black, etc.). In a previously described embodiment, the activity of the enzyme and/or another component present on the testing surface may cause the original color (e.g., green) to disappear (i.e., reduce significantly in saturation, brightness, or hue) such that the activated ABTS will transition from green to colorless upon detection of the presence of, for example, BPA. It will be appreciated that this reduction in saturation, brightness, or hue could be in the form of a continuous gradient, whereby the amount of decolorization corresponds to a relative concentration of the target substance.

It will also be appreciated that activated mediators other than ABTS and enzymes other than laccase may be used according to the principles described herein to test for the presence of many other analytes. In addition, it will be appreciated that more than one activated mediator and/or more than one enzyme may be used in combination to detect the presence of one or more analytes in a sample, where, depending on the desired result, the substrates may or may not interact with each other, other enzymes, and/or other components of the sample. It will be appreciated that the enzymatic reaction or reactions that may occur when a test sample is applied to the testing surface may or may not be connected directly with the downstream non-enzymatic reactions such as but not limited to the substrate or mediator reaction.

The preceding paragraph describes an exemplary method to extend the function of activated mediator assays through the use of multiplexing. As used herein, “multiplexing” will be understood to mean the use of one or more mediators, activated or not, and/or one or more enzymes, and/or one or more substrates in a single assay, reacting simultaneously or sequentially with one or more enzymes and one or more substrates where, depending on the desired result, the substrates may or may not interact with each other, other enzymes, or materials from the sample. Enzymes in a multiplexed assay may be any enzyme, protein or antibody.

Those skilled in the art will see that the combination of multiple mediators will affect the specificity as well as the detection limits of an analyte or analytes. The use of a mediator-substrate system may also be utilized to modify the functionality (such as the preferred solvent) or change the output color of the assay. Many mediators are also chromogenic in reaction to laccase or other appropriate enzyme, and/or other active mediator radicals so these mediators are also substrates. Some exemplary mediators include, but are not limited to, molecules that contain the basic structure contained in formula 1 above, where R1, R2, R3, and R4 can be any number of substitutions including but not limited to hydrogen(s), halogen(s), hydroxyl(s), aryl group(s), alkyl group(s), carboxylic acid(s), other phenolic moieties, and many others. Many mediators and substrates that react to ABTS radicals or appropriate enzymes are azo compounds with the basic structure represented by formula 3 below,

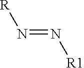

where R and R1 can be aryl (including substitutions) or alkyl. In addition, azine compounds having the basic structure shown in formula 2 above (e.g., ABTS, syringaldazine, and the like) are important in the reactions described herein.

Multiplexed detection systems as described herein may provide for a testing environment that restricts or eliminates the presence of substances/molecules that may interfere with the reactions, and may detect the presence of a chemical or chemicals of interest directly in one simple step. As such, while the possibility of false positive/negative results exists in many of the presently available conventional enzymatic assays, the methods and systems described herein advantageously remove the variables that may account for false positives and may rely on a single enzyme-based reaction test that directly determines whether the analyte of interest is present in a given sample or not.

In one approach, one or more control enzymes may be added that result in a positive or negative control to reduce the chance of false results. In another approach, one or more enzymes may be added as a control to determine that the components of the test are functioning as intended, e.g., confirm that the enzymes are active. For example, one or more enzymes may be added that may generate a predetermined color that indicates to a user that the test did not operate properly.

Multiplexing may be used to increase or decrease the number of detectable anaytes in a sample. By way of example only, even enzymes in the same family have different specificities and so they can be used to narrow or widen or change the specificity of the reactions. For example, two different forms of laccase may be used in a single assay to alter the properties of a reaction with mediators and/or substrates that are sensitive to laccase, such as, but not limited to, ABTS, Syringaldazine, and the analytes of interest. In one embodiment, using a combination of two or more enzymes and one or more substrates creates a multiplexed assay that will be selective for the target analyte while selecting against expected non-target analytes.

Rather than just change assay specificity, a multiplexed assay may be designed to physically eliminate interfering molecules, for example through use of specific protein kinases to remove the majority of proteins from a sample. Other enzymes can be used to remove antioxidants and diamines from the solution being analyzed by destruction or precipitation of such interfering molecules.

In one exemplary embodiment of multiplexing, the breadth of target analytes is increased by using one or more enzymatic reactions as a reporter to control the color output for colorimetric assays or as an intermediate electron carrier or as a current generating media. As used herein, “reporter” will be understood to mean any substance used to alert a user or a device to the changes occurring during an assay. Reporters may be chromatic, electronic, or utilize other methods of detection. An exemplary use of a reporter is to create a red-green binary output in an assay where the mediator selected to detect an analyte does not have this red-green output as a native feature of its chemistry.

In one embodiment, a binary result (or in some cases a gradient) can be generated using two separate reporter reactions: one in the direction of a positive result, and another in the direction of a negative result. As an example, a reporter can consist of a first reaction that changes from one color to another, including clear, upon reaction with an analyte of interest and a second, separate reaction that changes from one color to another, including clear, in the absence of an analyte of interest. This reporter is particularly useful for combining competitive binding antibody assays that change from clear to a color, for example red, in the presence of the target molecule with another reaction that will turn from clear to another color, for example green, in the absence of such target chemical. Alternatively, a reporter may consist of a first decolorization reaction combined with a second separate colorization reaction.

As another example, a reporter can consist of a decolorization reaction that reveals a second color hidden below the initial color. In this embodiment, the activated mediator covers a testing surface that is coated in a dye that is insoluble to the solution containing the analyte. In such a system, a target molecule or molecules may disrupt the stability of the activated mediator and thereby decolorize the radicals. Hidden beneath these colored radicals is another color that, when exposed, is visible to the user. One preferred embodiment is a red testing surface hidden by activated mediators that appear green to the user (indicating “safe”); in the presence of an analyte of interest, the green decolorizes, resulting in a red indication to the user (indicating “danger”). An alternate embodiment is a yellow testing surface hidden by activated mediators that are semi-transparent blue, resulting in a green indication to the user (indicating “safe”); in the presence of an analyte of interest, the blue decolorizes, resulting in a yellow indication to the user (indicating “caution”).

In some cases, the activity of one enzyme will decolorize or catalyze the products of its multiplexed enzymes to create a distinctly binary result or to create a variable color chart that may or may not be compared to a standard curve or known colors from previous trials. Specifically, a first enzyme may under certain conditions catalyze a first reporter reaction that transforms a first substrate from white or clear or any other initial color to green (indicating “safe”) and a second, different enzyme may under certain conditions catalyze a second reporter reaction that transforms a second substrate from white or clear or any other color to red (indicating “caution”). If the sample of interest does not contain the target analyte being tested for, the sample may be considered a negative sample (“safe”), and a color generally associated with “safe to proceed” (i.e., green) may be generated from the first reporter reaction. In this case, the red color of the second reporter reaction may be decolorized by reaction with the first enzyme; thus none of the product of the second reporter reaction would be visible and the result would be a visual indication of green.

Alternatively, if the analyte of interest is detected in the sample, the first enzyme may be inhibited completely by the interaction with the target analyte and thus none of the product of the first reporter reaction will be produced. In this case, the second enzyme may actively convert its product (which would normally be removed by the first enzyme), until the red color is clearly visible, thus indicating to the user an appropriate display of caution.

In one approach, if the enzymes used in the test have become inactive (e.g., due to prolonged storage or inadvertent contamination), or the pH, temperature, denaturants, or any other direct or indirect component and/or inhibitor of the basic reactions involved in the test are out of workable range, then a reporter reaction may be used to decolorize the indicator color, or to generate some other appropriate cautionary color, alerting the user that the test was invalid. For example, the test may be invalid if one or more reagents in the test have gone bad, or if a contaminant in the sample inhibited the enzymes that need to be active for the test to properly function. A specific color-coded indication of a test that failed advantageously prevents the generation of false test results and allows the user to run another test to obtain a correct result.

It will be appreciated that the red/green output described herein is an example only, and any other colors may be used as appropriate indicators. Preferably, the enzymes used in reporter reactions are not inhibited by any of the reagents/components in the sample unless the assay is designed to alert the user that this reagent or component is present in the testing medium.

A test device for multiplexed assays may be configured such that the enzymes and the substrates are placed separately and/or sequentially onto one or more testing surfaces and dried, but any method of application of the active ingredients onto the testing surface(s) may be used, and may include inkjet-like printing of components onto a testing surface. Alternatively, the enzymes can be lyophilized and powdered and rubbed onto a testing surface that has already been coated in reactive intermediates, mediators, or substrates and dried, advantageously allowing for a single testing surface or membrane to be used instead of two or more membranes common in more complex enzyme assays. The enzymes may be rubbed into a testing surface dry, or may be mixed with an inert mordant in order to facilitate the rehydration and reaction rates. The testing surface may be wrapped around the substrate layer and placed inside, for example, a test tube such that when a sample to be tested is introduced into the test tube, the test reaction will be initiated to generate a result indicating the presence of the analyte.

In one exemplary application, the present methods and systems may be used to detect the presence and/or concentrations of testosterone (or other steroid or non-steroid hormones) in the blood stream. In such a test, two or more enzymes that interact with testosterone and sex hormone binding globulin (SHBG) may be used. The assay may be run as a liquid, e.g. in a single well, on a testing surface, or other. The test results may be compared to a standard curve that would advantageously provide for a one-step determination of total and free testosterone in a patient's blood relatively easily as compared to the conventional methods of using ELISA to detect each chemical and then combine the results to estimate the probable amount. The present methods and systems may thus allow users to quickly and easily check their testosterone levels at home, similar to the way glucose levels are tested.

In one approach, a testing surface including an activated mediator specific to testosterone may include one or more components that would provide a binary result in the faun of an appropriate color when the detected testosterone levels are low (i.e., below 300 mg/dl) or normal (i.e., between 300 mg/dl and 1000 mg/dl). For example, the activated mediator (or another reagent) used in the test for testosterone may be configured to provide a color change to red when testosterone levels below 300 mg/dl are detected and configured to provide a color change to green when testosterone levels above 300 mg/dl are detected. The binary result may thus provide the user with an easy to understand color indication that the user may compare to a color chart relating to expected testosterone levels for an age group.

In another exemplary application, the present methods and systems may be used to detect the presence of substances commonly associated with drink tampering. Drink tampering refers to the purposeful addition of substances to a person's drink with the intent of causing harm; the usual intent is to render a person's inhibitions ineffective or render a person entirely unconscious. Common substances known to be used to tamper with drinks include, but are not limited to, benzodiazpines such as Alprazolam (Xanax), flunitrazepam (Rohypnol), or many others), sleeping medications such as zolpidem (Ambien), GABA derivatives such as 4-Hydroxybutyrate (GHB), and y-butyrolactone (GBL), ketamine (K), sedative analgesics such as morphine, oxycontin, or fentanyl, derivatives of barbituric acid, nonbenzodiazepines, and methaqualone and its analogues.

The substances provided as examples above are a chemically diverse group of molecules that cannot be detected in one test and in one step using the presently known methods and systems. In one approach, one or more of these substances could be detected using an activated mediator alone, such as ABTS, or an activated mediator and a substrate with the proper structure by adding stronger mediators (e.g. (2,2,6,6,-Tetramethylpieridin-1-yl)oxy, also known as TEMPO) to broaden the specificity of the test.

A preferred embodiment for detecting multiple drink tampering substances using the present methods and systems described herein is to multiplex all of the competitive binding antibodies for the substances of interest into one test which will proceed from clear to red in the presence of an analyte of interest. In such an approach, the binary indication may be created using two different reactions that occur simultaneously. Specifically, the competitive binding antibodies and related components may react to indicate the presence of an analyte and another set of enzyme(s) and substrate(s) may react towards the negative indication, which would preferably be green or any other desired color.

Multiplexing the competitive binding antibodies for substances of interest, combined with appropriate reporters using the methods, devices, and systems described herein can also be used to create tests for, as examples, animal proteins (useful for vegetarians), salmonella, human immunodeficiency virus (HIV), hepatitis C (HEP-C), and many other substances that are of interest to a large number of consumer and industrial users.

It will be appreciated that the methods and systems described herein may be used to successfully detect the presence of and the levels of not only BPA, heavy metals, testosterone, and drink tampering substances, the detection of which has been discussed above by way of example only. The methods, devices, and systems described herein may also be used to create an instant test platform for a wide range of target analytes including, but not limited to, BPS and other polyphenols, simple phenols, lignin, dihydrobenezene and their derivatives, and antioxidants; peanut, gluten, grain, or other allergens in foods; hormones, antibodies and/or pathogens and/or DNA/RNA in blood and/or sputum and/or urine, and/or sweat; and for the presence of chemical compounds or contaminants in environmental samples, water, other drinkable beverages, food, and the like.

One advantage of the methods and systems described in the present application is that, when used for detection of substances in a predefined medium/solution, the composition of the testing solution used to react with the sample being analyzed may be modified to provide a wide variety of activated mediators having different specificity, which permits the assay designer to design a variety of systems that can selectively detect different substances based on the solution on the testing device. Another advantage is that the per-test cost of the methods, devices, and systems described in the present application is low due to the absence of expensive and complicated analytical machines such as spectrophotometers, and the methods and systems as described in the present application may be amenable to in-home use. Yet another advantage of the systems and methods as described herein is that generation of a binary-type result that provides a visual indication easily understandable to the user. Still another advantage of the systems and methods described herein is that, due to the decoupling of the activated mediator from the enzyme and the preservation of the activated mediator separately from the enzyme, the test results according to the methods and systems described herein are generated significantly faster as compared to the conventional multiplexing assays.

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept.

1. A method for detecting one or more predetermined analytes in a sample, the method comprising providing at least one activated mediator configured to produce a detectable change upon reaction with the one or more predetermined analytes in the sample.

2. The method of claim 1, further comprising:
combining one or more enzymes with one or more mediators to form an aqueous solution; and
reacting the one or more enzymes with the one or more mediators to form an aqueous solution including the at least one activated mediator in radical form.

3. The method of claim 2, further comprising inactivating the one or more enzymes contained in the aqueous solution including the one or more mediators in radical form prior to further reaction.

4. The method of claim 2, further comprising separating the one or more enzymes contained in the aqueous solution including the one or more mediators in radical form from the aqueous solution.

5. The method of claim 2, further comprising drying the aqueous solution including the one or more mediators in radical form onto a powder carrier.

6. The method of claim 2, further comprising drying the aqueous solution including the one or more mediators in radical form into powder form.

7. The method of claim 2, further comprising incorporating the solution into a testing surface by depositing at least a portion of the aqueous solution including the at least one activated mediator onto a testing surface and drying the testing surface.

8. The method of claim 7, wherein the testing surface includes a test strip produced from low lignin paper.

9. The method of claim 7, further comprising covering at least a portion of the testing surface with at least one protective material including at least one of polyethylene, polypropylene, polystyrene, polycarbonate, aluminum, and combinations thereof.

10. The method of claim 7, further comprising applying a sample liquid to the portion of the testing surface including the activated mediator by swabbing the portion of the testing surface including the at least one activated mediator with a material at least in part wetted with the sample liquid.

11. The method of claim 1, wherein the detectable change upon reaction of the at least one activated mediator with the one or more predetermined analytes is in the form of one of a visual colorimetric indication and a detectable change in electrical properties.

12. The method of claim 11, wherein the visible colorimetric indication in response to a reaction of the at least one activated mediator with the one or more predetermined analytes in the sample further comprises generating a visible color change of the at least one activated mediator from a first color to a second color in response to detecting a predetermined minimum concentration of the one or more predetermined analytes in the sample.

13. The method of claim 11, wherein the visible colorimetric indication in response to a reaction of the at least one activated mediator with the one or more predetermined analytes in the sample further comprises generating a color of a first saturation, brightness, and hue when a concentration of the one or more predetermined analytes below a first threshold is detected, generating a color of a second saturation, brightness, and hue when a concentration of the one or more predetermined analytes above the first threshold and below a second threshold is detected, and generating a color of a third saturation, brightness, and hue when a concentration of the one or more predetermined analytes above the second threshold and below a third threshold is detected.

14. The method of claim 11, wherein the visible colorimetric indication in response to a reaction of the at least one activated mediator with the one or more predetermined analytes in the sample further comprises generating a certain gradation in the saturation and/or brightness and/or hue of a color that corresponds to a relative concentration of the one or more predetermined analytes in the sample.

15. The method of claim 11, wherein the visible colorimetric indication in response to a reaction of the at least one activated mediator with the one or more predetermined analytes in the sample further comprises generating a color indicating to a user that one or more reagents on the testing surface failed to function properly.

16. The method of claim 2, wherein one of the one or more mediators is 2,2′-azino-bis(3-ethylbenzothiazoline-6-sulphonic acid) (ABTS), wherein one of the one more enzymes is a laccase obtained from at least one of Trametes Versicolor, Pleurotus Ostreatus, Pleurotus Species, and a recombinant laccase, and wherein the at least one activated mediator is ABTS radical cation.

17. The method of claim 1, wherein the at least one activated mediator is ABTS radical cation and the one or more predetermined analytes is selected from a group consisting of chemical compounds, amino acids, pathogens, nucleic acids, metals, and combinations thereof.

18. The method of claim 17, wherein the one or more predetermined analytes is bisphenol A (BPA), and wherein the ABTS radical cation changes color from green to red in response to reacting with the BPA in the sample.

19. A testing surface for detecting a predetermined analyte in a sample, the testing surface including an enzyme-activated mediator applied onto at least a portion of the testing surface, the enzyme-activated mediator being visible on the at least a portion of the testing surface to a user in a first color prior to application to the at least a portion of the testing surface including the enzyme-activated mediator of a sample, the enzyme-activated mediator being configured to undergo visible color change to a second color different from the first color in response to the sample applied to the at least a portion of the testing surface including the enzyme-activated mediator including a predetermined analyte reactive with the enzyme-activated mediator.

20. A kit for detecting a predetermined analyte in a sample, the kit including:
a testing surface including an enzyme-activated mediator applied onto at least a portion of the testing surface, the enzyme-activated mediator being visible on the at least a portion of the testing surface to a user in a first color prior to application to the at least a portion of the testing surface including the enzyme-activated mediator of a sample, the enzyme-activated mediator being configured to undergo a visible color change to a second color different from the first color in response to the sample applied to the at least a portion of the testing surface including the enzyme-activated mediator including a predetermined analyte reactive with the enzyme-activated mediator; and
a swab configured to absorb a portion of the sample and apply the absorbed portion of the sample to the at least a portion of the testing surface including the enzyme-activated mediator; and
a packaging material separating and enclosing the testing surface and the swab.
